(19) United States
(12) Patent Application Publication
(10) Pub. No.: US 2010/0228126 A1
(43) Pub. Date: Sep. 9, 2010

(54) ULTRASOUND TREATMENT AND IMAGING APPLICATOR

(75) Inventors: Charles D. Emery, Sammamish, WA (US); Barry Friemel, Redmond, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: MIRABILIS MEDICA INC., Bothell, WA (US)

(21) Appl. No.: 12/719,782

(22) Filed: Mar. 8, 2010

Related U.S. Application Data
(60) Provisional application No. 61/158,295, filed on Mar. 6, 2009.

(51) Int. Cl. A61N 7/00; A61B 8/14

(52) U.S. Cl. 600/439

(57) ABSTRACT

An ultrasound treatment system includes an applicator in which a therapeutic ultrasound transducer is surrounded by an annular imaging transducer. Illumination signals generated by the therapy or imaging transducer are sequentially or simultaneously delivered to tissue in a viewing space to create corresponding echo signals that are received by the elements of the annular imaging transducer. These echo signals are analyzed with a processor to produce an image of tissue in the viewing space.

### CROSS-REFERENCE TO RELATED APPLICATION

This application claims the benefit of U.S. Provisional Patent Application No. 61/158,295 filed Mar. 6, 2009, which is herein incorporated by reference in its entirety.

### BACKGROUND

Uterine fibroids, which are benign tumors in the muscle wall, are a common health problem in women and can occur in various regions of the uterus. Fibroids are the most common benign neoplasm occurring in women of reproductive age, affecting 16 million women in the United States. Approximately 25% of all women that have fibroid tumors exhibit clinically-significant symptoms such as heavy and irregular menstrual bleeding, pelvic cramps, increased urinary frequency, and infertility.

The most common current treatment option for the treatment of fibroids is hysterectomy, which involves the complete removal of the uterus. Typically, one out of every two hysterectomies performed in the United States are due to presence of fibroid tumors. Hysterectomy is not a reasonable option for women wishing to retain fertility. Despite its invasiveness, long recovery times and other drawbacks, approximately 50% of women diagnosed with fibroid tumors in the United States get a hysterectomy.

Myomectomy, which is a surgical procedure to remove the fibroid while leaving the uterus intact, has similar risks to a hysterectomy but with less risk to fertility. Uterine artery embolization (UAE) attempts to destroy a fibroid through selective ischemic injury. UAE procedures have been shown to have limited efficacy and may have adverse effects on other organs because of poor localization. Hormone therapy is another option for patients. However, pharmacological intervention is costly, has potential side effects and must be used continuously to prevent re-occurrence of symptoms.

A more recent treatment option is the use of MRI-guided focused ultrasound surgery (MRgFUS). However MRgFUS has shortcomings that include exorbitant capital costs, referral pattern issues, and lengthy procedure times. MRI systems cost over $1 million and the complexity of an MRI compatible HIFU system increases the total capital cost to over $2 million. Procedures may be three to four hours and require multiple physicians such as a gynecologist and radiologist.

Ultrasound-guided HIFU (USgHIFU) systems are intended to offer the benefits of non-invasive treatment, but without the drawbacks of MRgFUS, such as high cost and limited access. USgHIFU does so by using ultrasound imaging to target and treat the fibroid.

Most commonly proposed ultrasound-guided HIFU systems use a separate imaging transducer inside the HIFU aperture to optimize system performance. This strategy creates a conundrum for space in the treatment aperture. Reducing the HIFU aperture area may lessen the ability to therapeutically treat whereas reducing the imaging aperture may limit the ability to visualize the target tissue and surrounding tissues. One example is the placement of an imaging array in the middle of the therapy device. This approach reduces the possible aperture space available for the therapy transducer material and affects therapy beam performance due to the presence of the central hole in the aperture. It also physically couples imaging and therapy apertures at the array level.

Another proposed solution is to design a transducer with elements that can do both imaging and supply therapy. These dual mode ultrasound arrays (DMUAs) have limited capability because of the trade-offs between the imaging and therapy requirements. For example, imaging requires wide bandwidth, higher frequency operation whereas HIFU therapy requires high average power with narrowband, low frequency operation.

Given these problems, there is a need for an ultrasound treatment system with a combination applicator having transducers that can deliver therapy signals to the patient and receive ultrasound signals in order to image the tissue. The imaging elements should occupy minimal space and not affect the ability of the therapy elements to deliver treatment signals. In addition, the transducer should be able to create volume images and C-planes (imaging planes parallel to transducer face) for easy and fast interpretation and tracking of the target and surrounding tissues, detect obstacles (e.g. bone, bowel, air) in the therapy beam path, assess therapy beam distribution and evaluate the target before, during and after treatment.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

To address the problems discussed above and others, the technology disclosed herein is an ultrasound treatment system with an applicator that can both deliver therapy and detect echo signals. The applicator includes a therapy transducer that has a mechanically or electronically adjustable focus and steering direction that can be selectively moved or broadened to transmit illumination signals into a viewing space that includes a tissue volume to be treated. An imaging transducer surrounds an outer portion of a therapy transducer. In one embodiment, the therapy transducer provides illumination signals that are delivered into the viewing space to produce corresponding acoustic echo signals. The imaging transducer has a number of elements that receive the acoustic echo signals and produce corresponding electronic echo signals. A processor is programmed to selectively combine the electronic echo signals to produce an image of the tissue in the viewing space.

In one embodiment, the imaging transducer comprises an annular ring of a number of receive elements each having at least one dimension that is less than a wavelength of the illumination signals produced by the therapy transducer.

In yet another embodiment, the applicator includes a second annular imaging array of transducer elements that are oriented to capture acoustic echo signals in a cylindrical volume surrounding tissue that is insonified by the therapy transducer.

In yet another embodiment, the imaging transducer may include one or more higher power transmit elements to produce illumination signals that are delivered to the tissue. The transmit elements may be in fixed position or rotatable around the receive elements.

In yet another embodiment, the therapy transducer may be used to produce a push signal for elasticity or shear wave imaging.

In still another embodiment, an applicator includes two or more annular imaging arrays, wherein the transmit elements of one of the annular imaging array are laterally displaced or mechanically or electrically focused to provide a single virtual source of ultrasound signals that is displaced from a skin surface.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram of an ultrasound imaging and therapy system in accordance with an embodiment of the disclosed technology;

FIG. 2A illustrates an applicator with a therapy transducer and a surrounding imaging array in accordance with an embodiment of the disclosed technology;

FIG. 2B illustrates how signals from the receive elements on an annular imaging array are combined to calculate a value for one point in a target volume to be imaged in accordance with one embodiment of the disclosed technology;

FIG. 2C illustrates an applicator with a therapy transducer and two annular imaging arrays in accordance with an embodiment of the disclosed technology;

FIG. 2D illustrates an applicator with a therapy transducer and an annular imaging array that includes one or more higher power elements in accordance with an embodiment of the disclosed technology;

FIG. 3A illustrates a viewing space imaged by an annular imaging array and an illumination signal from a therapy transducer;

FIG. 3B illustrates an illumination signal created by an annular array and a cylindrical image captured by a second annular imaging array;

FIG. 3C illustrates an illumination signal produced from a therapy transducer and a conical image captured by an annular imaging array with receive elements that are oriented to focus on the outside of a focal zone of the therapy transducer; and

FIGS. 4A and 4B illustrate alternative techniques to increase the level of illumination signal that can be delivered to tissue from an annular array in accordance with another embodiment of the disclosed technology

### DETAILED DESCRIPTION

As indicated above, the technology disclosed herein relates to an ultrasound treatment system with a combination applicator that both delivers therapeutic energy to the patient and receives ultrasound signals to image the tissue in the body. In the embodiments described below, the therapy delivered is high intensity focused ultrasound or HIFU. However it will be appreciated that the disclosed technology can also use non-focused ultrasound energy for treating tissue.

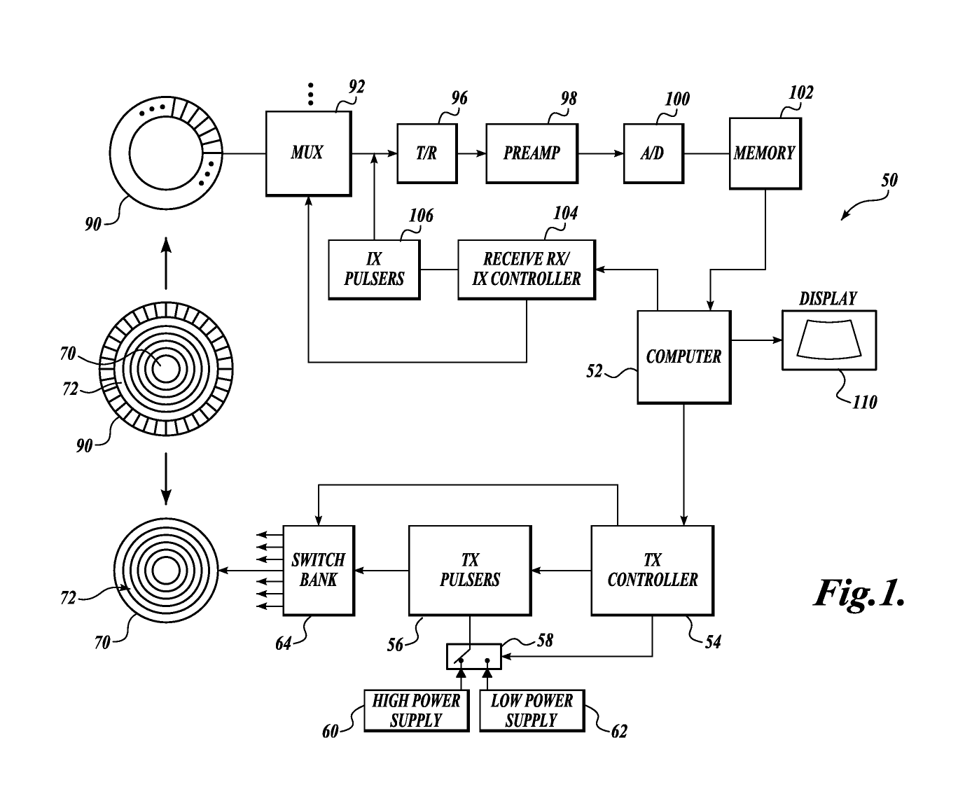

One embodiment of a system in accordance with the disclosed technology is shown in FIG. 1. As shown, the system 50 includes a computer system 52 with one or more processors that are configured to execute a sequence of program instructions in order to implement the functions and methodology described below. The instructions are stored on a non-transitory computer readable media such as a hard drive, CD-ROM, DVD, flash drive, volatile or non-volatile memory or an integrated circuit etc.

The computer system interacts with a physician through an input mechanism such as keyboard, mouse, stylus pen, touch screen etc. so that the physician can indicate a volume of tissue to be treated. The computer system 52 provides the coordinates of a desired treatment volume of tissue that a physician would like to treat to a transmit controller 54. The transmit controller 54 is an electronic device that operates to determine parameters such as the timing, amplitude and phase of one or more driving signals that should be delivered to a therapy transducer in order to direct treatment energy to the desired target. The transmit controller may also operate to produce commands to electronically or mechanically move the focus of the therapy transducer. The transmit controller may also configure the therapy transducer in order to illuminate tissue for imaging and/or targeting. This is accomplished by applying the correct phase and amplitude on the therapy transducer. The details of the transmit controller 54 are considered known to those of skill in the art and therefore are not discussed further.

The outputs of the transmit controller 54 are supplied to a transmit pulser 56 that produces the ultrasound driving signals in response to the signals from the transmit controller 54. In one embodiment, the transmit pulser 56 is connected to either a high power supply 60 or a low power supply 62 through a switch 58. In one embodiment, the switch 58 is controlled with signals from the transmit controller 54. The power supplies 60 and 62 are selected depending on whether the signals to be produced by the therapy transducer are high power therapy signals such as may be used when actively treating tissue, or monitoring the harmonic content of a received echo signal in order to adjust treatment power or to control treatment time, or when used for elasticity imaging. High power or low power signals can be selected to create illumination signals for imaging or other uses. If a power supply can change its power output fast enough and has enough dynamic range, a single high/low power supply can be used. The transmit pulser 56 provides the driving signals to a switch bank 64 that directs the driving signals to one or more elements of a therapy transducer 70.

The therapy transducer 70 is preferably a fixed or variable focus transducer that can be mechanically or electronically controlled such that illumination signals produced are directed over a viewing space. A fixed focus transducer can be mechanically moved by servo motors or the like so that tissue in the viewing space is illuminated as the focal zone of the transducer is moved. If an electronically controllable transducer is used, the focal zone of the transducer may be electronically moved to sequentially illuminate the tissue in the viewing space or the focal zone can be de-focused to broaden the signals transmitted such that some or all of the tissue in the viewing space is simultaneously illuminated. One example of an electronically controllable therapy transducer is an annular and/or sectored ultrasound transducer that is controllable to selectively deliver high intensity focused ultrasound (HIFU) or non-focused ultrasound pulses to the tissue of a patient. If the focus of the therapy transducer 70 is electronically controllable, then the configuration of the switch bank 64 is controlled by the transmit controller 54 such that the driving signals are applied to all or a subset of the elements of the transducer as necessary to adjust the focus or illumination region of the signals produced by the therapy transducer as desired. The details of the transmit pulser 56 and the switch bank 64 as well as the design and construction of the therapy transducer 70 are known to those or ordinary skill in the ultrasound arts.

To produce an image of tissue within a viewing space that includes the target tissue volume (e.g. a fibroid tumor) to be treated and the surrounding tissue including tissue between the therapy transducer and the target tissue volume, the applicator includes an annular imaging array 90 that is located circumferentially around the therapy transducer 70. The annular imaging array 90 is modular such that it may be mechanically and electrically independent of the therapy transducer 70. Because the annular imaging array 90 is on the outside of the therapy transducer, it is easy to make electrical connections to the receive elements. In addition, the receive elements and transmit elements of the applicator can be individually controlled. In the embodiment shown, the annular imaging array 90 comprises a number of sectored piezoelectric receive elements wherein each element has the appropriate directivity or acceptance angle to receive signals of the scattered energy from the illuminated viewing space (e.g. at least one dimension that is smaller than the wavelength of the signals produced by the therapy transducer 70 or are mechanically shaped or lensed to receive the scattered energy from the viewing space). In one embodiment, the annular imaging array 90 includes 512 receive elements that are located around the circumference of the therapy transducer 70.

The piezoelectric elements of the annular imaging array are generally too small to produce enough acoustic power to produce echo signals with a sufficient signal to noise ratio to be able to produce an image of the tissue in the viewing space. Therefore the focus of the therapy transducer is adjusted to produce illumination signals that are sequentially or simultaneously transmitted into the viewing space. If the tissue is to be treated after imaging, the focus of the therapy transducer is then adjusted to concentrate the ultrasound signals produced to treat the desired treatment volume.

In this embodiment, the elements of the annular imaging array 90 produce electronic signals in response to detecting the acoustic echo signals that are created by the delivery of illumination signals into the tissue by the therapy transducer 70. Because there may be more receive elements in the annular imaging array than the number of channels in the receive electronics, the signals from the annular imaging array may be processed in groups. In the embodiment shown, a number of multiplexers 92 are provided to select signals from the receive elements of the annular imaging array 90. In one embodiment, each multiplexer 92 selects one of eight input lines each of which is connected to one receive element in the annular imaging array. In the exemplary embodiment shown, if there are 512 receive elements in the annular imaging array and each multiplexer can select one of eight receive elements, then 512/8 or 64 multiplexers are required to receive all the signals. Depending on the speed with which the multiplexers 92 can be switched, more than one illumination signal or signals may be required in order to obtain signals from each of the receive elements in the annular imaging array.

The outputs of the multiplexers 92 are supplied through a transmit/receive switch 96 to a multi-channel pre-amplifier 98 that boosts the level of the signals received from the annular imaging array and may perform additional signal conditioning. The outputs of the pre-amplifier 98 are fed to an analog to digital converter 100 that converts the analog electronic echo signals to a corresponding digital form for storage in a memory 102. The memory 102, which may be part of the computer system 52, is readable by the computer system 52 or other special purpose digital signal processor that executes a beam forming process on the digitized receive signals to determine one or more of the amplitude, power and/or phase of the received signals for a region in the tissue. The beam formed signals can be used to produce an image of tissue in the viewing space into which the illumination signals are delivered on a display 110. Alternatively, the image can be stored on a computer readable media (hard drive, DVD, video tape etc.) or transmitted to a remote computer over a wired or wireless communication link.

To image the tissue in the viewing space, the therapy transducer 70 produces one or more illumination signals that interact with the tissue in the viewing space to create the echo signals that are detected by the receive elements of the annular imaging array. The TX controller configures the switch bank 64 so that the driving signals are applied to the desired elements of the therapy transducer 70 to sequentially or simultaneously illuminate the tissue in the viewing space. The TX controller 54 selects the appropriate power supply via the switch 58. The amplitude and timing of the driving signals are determined by the TX controller 54 depending on the size and/or location of the viewing space to be imaged and the TX controller signals the TX pulsers 56 to deliver the driving signals to the desired elements of the therapy transducer.

Prior to transmitting the illumination signals into the viewing space, the computer system 52 configures the receive electronics to detect the echo signals from the tissue in the viewing space. A receive controller 104 (described below) sets the position of the multiplexers 92 depending on which elements of the annular imaging array are to be connected to the receive electronics. The transmission of the illumination signals and configuration of the multiplexers 92 and the receive electronics is therefore coordinated.

By using the therapy transducer 70 to generate the illumination signals, sufficient signal power is applied to allow the receive elements of the annular imaging array to produce echo signals that can produce images of the tissue.

In some embodiments described below, selected elements of the annular imaging array can also be used to transmit illumination signals into the viewing space. In this case, the system includes a receive/transmit controller 104 and a number of transmit pulsers 106. When the receive controller is used to control the transmission of signals, it can be referred to as an “IX controller” that refers to the fact that the elements of the imaging array (I) are used to deliver illumination signals to the tissue. When elements of the annular imaging array are being used to receive signals, the receive controller 104 sets the positions of the multiplexers 92 so that the correct receive elements are connected to the pre-amplifier 98 and the A/D converter 100. If one or more of the elements of the imaging array produce illumination signals, the transmit controller 104 supplies parameters for the driving signals to be produced by the transmit pulsers 106 and delivered to the elements of the annular imaging array under the direction of the computer system 52.

In one embodiment, the power of the treatment pulses used to treat a target volume is adjusted as a function of the harmonics in the echoes that are created from the treatment pulses. Therefore the design (e.g. size, acoustic materials etc.) of the receive elements in the annular imaging array should be selected so that they are sensitive to the expected frequency of the harmonics. The therapy transducer may also be excited so that the illumination signals are at a different frequency than the frequency used for the treatment signals in order to better match the performance of the receive elements. If the size of the receive elements in the annular imaging array is small, the focal zone of the annular imaging array can be electronically moved over the volume of tissue in which the illumination signals produced by the therapy transducer are transmitted.

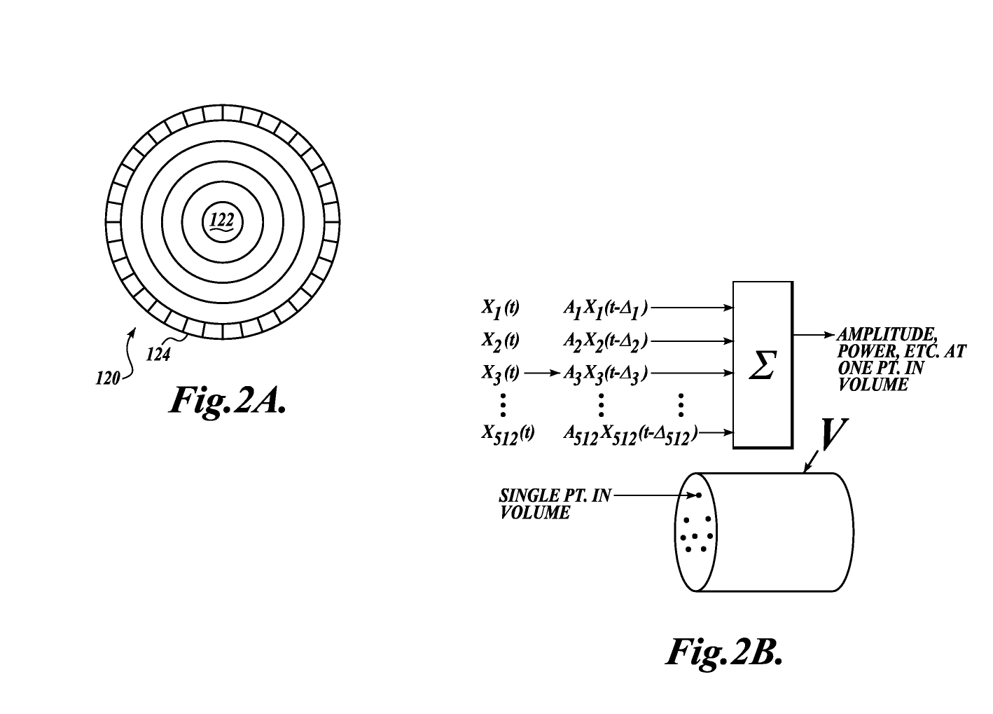

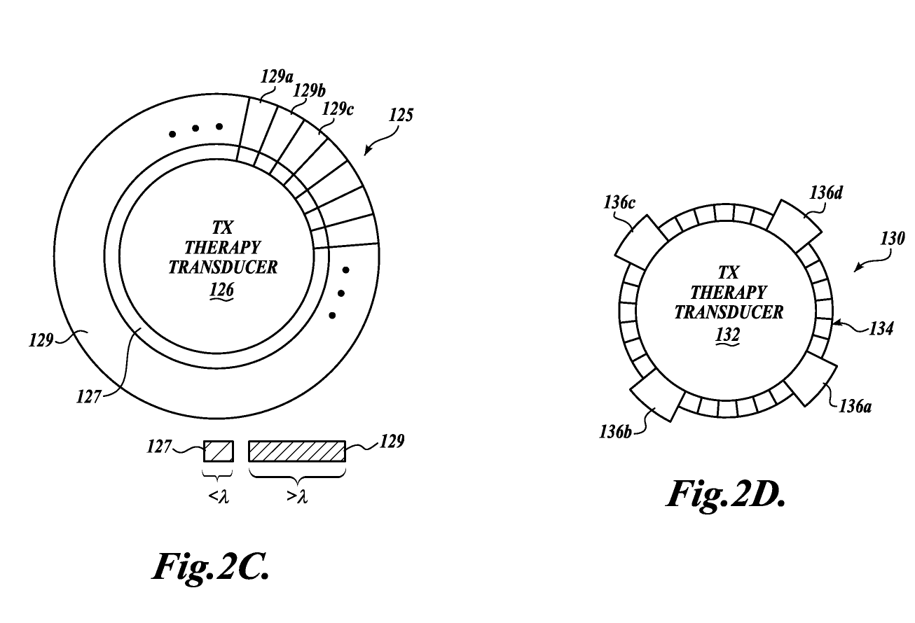

FIGS. 2A, 2C and 2D illustrate different embodiments of an applicator that includes an imaging and a therapy transducer in accordance with the disclosed technology. In FIG. 2A, an applicator 120 has a centrally located therapy transducer 122 that is surrounded by an annular imaging array of receive elements 124. In one embodiment, the therapy transducer 122 has a number of annular rings that are used to adjust the focus of the transducer. Although a therapy transducer with annular rings is illustrated, it will be appreciated that other configurations such as a sectored therapy transducer could also be used. In one embodiment, the therapy transducer and the receive elements of the annular imaging array are designed to operate in the same frequency range. In other embodiment, the size of each of the elements 124 in the annular imaging array is smaller than the wavelength of the signals produced by the therapy transducer 122 to illuminate the tissue so that the receive elements are sensitive to harmonics of the signals produced from the therapy transducer.

When imaging a volume of tissue, signals from the annular imaging array can be processed in adjacent groups. For example, if 512 elements are present and processed in groups of 64 elements at a time, elements 1-64 can be processed in response to one illumination pulse or pulses followed by elements 65-128 etc.

FIG. 2B illustrates a technique in accordance with one embodiment of the disclosed technology to produce an image of a tissue volume, V, using the echo signals detected by the receive elements of the annular imaging array. The receive electronics operate to detect digitized echo signals x1(t), x2(t), x3(t) . . . X512(t) from each of the receive elements of the annular imaging array. The signals are weighted with an apodization constant and delayed by the computer system or other programmed processor depending on: the directivity of the transmit source, the acceptance angle of the receive element, the distance in the aperture between the transmit source and receive element, the distance between the transmit source and the interrogation point in the volume and then from the interrogation point in the volume to the receive element. The weighted and delayed signals from each receive elements are summed in order to calculate an amplitude, power or other signal characteristic for the point in the volume. Multiple transmit sources may be used to create a synthetic aperture image and/or for image compounding. The next point in the volume is selected and the process repeats for all points in the volume.

In some situations, it may be advantageous to image a cylinder of tissue that encompasses the region that will be insonified by the treatment signals produced by the therapy transducer. For example, if a cylinder of tissue is imaged and no gas, bowel, bone or other non-desired tissue is present within the cylinder than it may be safe to treat the tissue within the cylinder with high power HIFU signals. Such imaging may also aid in detecting any problems with acoustic coupling of the HIFU beam to the tissue surface (e.g. areas of high reflection at the tissue surface may indicate poor coupling). In the embodiment shown in FIG. 2C, an applicator 125 includes an inner therapy transducer 126, a first annular imaging array 127 and a second annular imaging array 129. The therapy transducer 126 and the first annular imaging array 127 are as described above.

The second annular imaging array 129 preferably includes a number of piezoelectric elements 129a, 129b, 129c etc. that produce electronic signals in response to received acoustic echo signals. The receive elements of the first and second annular imaging arrays may be connectable with switches or the like so that signals from the receive elements of either or both imaging arrays can be detected. In the embodiment shown, the size of the receive elements 129a, 129b, 129c, in the second annular imaging array are larger than those of the first annular imaging array to make them more sensitive to received echoes. However such larger elements have a reduced ability to detect off angle signals. Therefore the receive elements of the second annular imaging array are more sensitive to a region that is directly ahead of the receive elements. By orienting the receive elements of the second annular imaging array towards an area that surrounds the area of the volume of tissue to be treated, an image of the tissue in a cylinder can be created. As will be appreciated however, the direction of maximum sensitivity of the elements in the annular imaging transducer 129 can be varied by changing the orientation of the elements such as by mounting them on a form or so forth. In the embodiment shown in FIG. 2C, the second annular imaging array 129 is configured to produce an image of a cylinder surrounding the tissue to be treated with the therapy transducer. The illumination signals can be produced either by the therapy transducer 126 or by the elements of the first or second annular imaging array. To produce a cylindrical image, overlapping sections of the receive elements are processed sequentially such as elements 1-64 followed by elements 2-65, 3-66, 4-67 etc. if the illumination signals are produced by a group of elements focused from the annular ring.

In some instances, the size of the elements in the first or second annular imaging array may be too small to generate enough signal power to produce echoes with a good signal to noise ratio. Therefore one or more “piston” elements can be incorporated into the annular imaging array. In the embodiment shown in FIG. 2D, an applicator 130 includes a central therapy transducer 132, and a first annular imaging array 134 that includes a number of smaller receive elements. In addition, the annular imaging array 134 includes a number of higher power transmit piston elements 136a-136d. The piston elements 136 are designed to transmit higher power illumination signals into the tissue so that the remaining elements of the imaging array can produce electronic echo signals with sufficient signal to noise ratio so that images of the underlying tissue can be produced.

In one embodiment, the transmit piston elements 136a-136d are larger than the receive elements so that the acoustic power they can transmit is larger than can be transmitted from the receive elements. The transmit elements may be incorporated into the same array as the receive elements or may be incorporated into a separate array such as a second annular array that is located around the circumference of the array in which the receive elements are located. In another embodiment, one or more transmit elements 136 are mechanically moveable around the array of receive elements on a spinning mechanism such that a single transmit element can illuminate the viewing space. Alternatively two or more transmit elements can be mounted to a mechanism that moves the transmit elements back and forth around the circumference of the receive array to illuminate the viewing space. It is also possible to construct the annular imaging array as one or more receive elements that are rotatable around the therapy transducer. The receive elements and the transmit elements can be individually controlled and may be asynchronously moved.

To further increase the signal to noise ratio, the transmit elements 136 may employ temporal or spatial coding of the illumination signals.

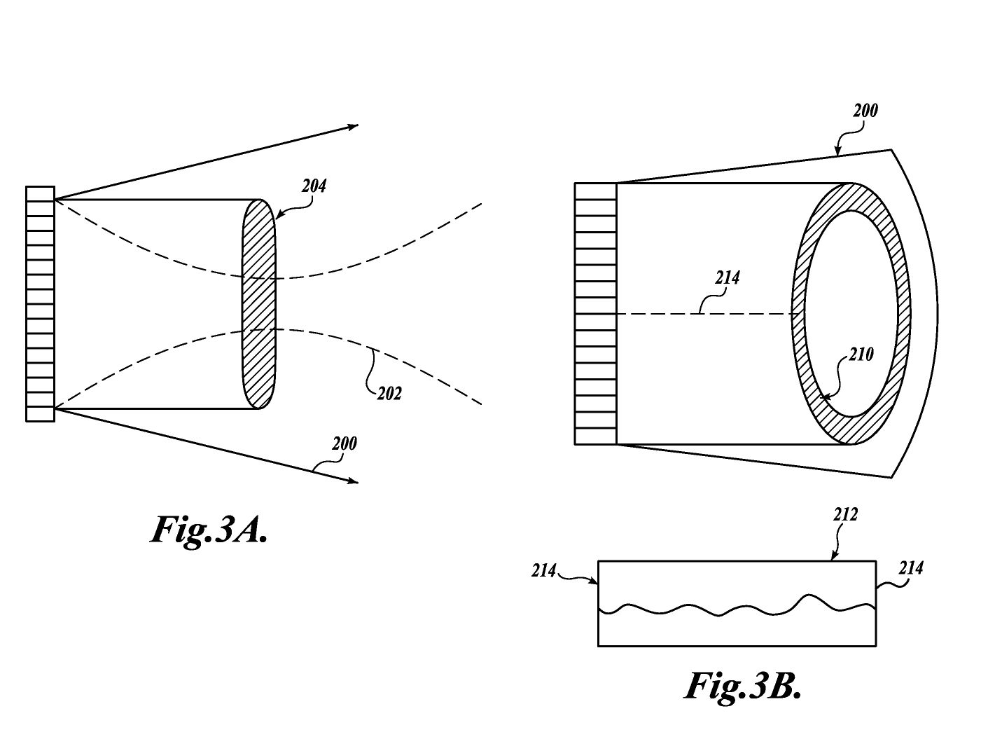

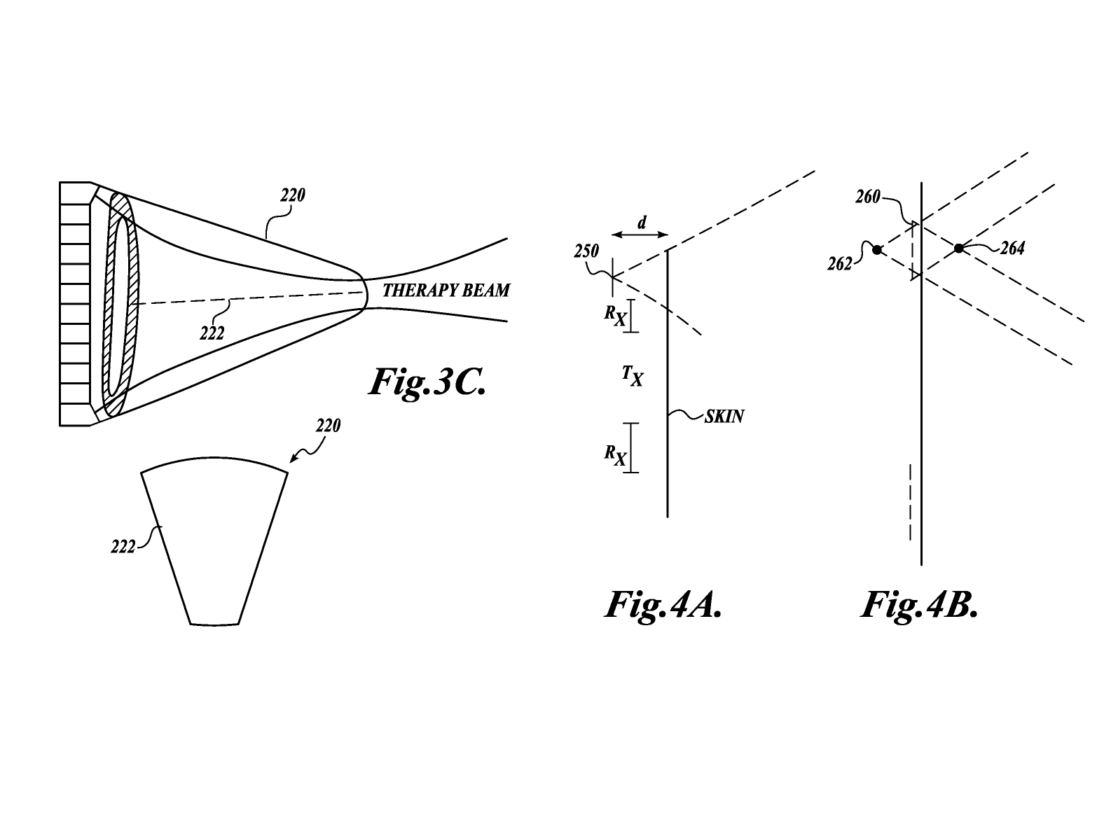

FIG. 3A illustrates an illumination signal 200 that is produced by the therapy transducer. The illumination signal 200 can be produced at either a lower power or a therapeutic power. The focus of the therapy transducer is adjusted so that the tissue in the viewing space is sequentially or simultaneously illuminated. The annular imaging array surrounding the therapy transducer receives echo signals created in response to the illumination signal(s) and produces corresponding electronic echo signals which are used to produce an image of a tissue volume 202 in the viewing space. If tissue in the viewing space is to be treated, the focus of the therapy transducer is changed to concentrate the ultrasound signals into a portion of the desired treatment volume in order to treat the tissue.

In the example shown in FIG. 3B, an annular imaging array is used to produce a cylindrical image 210 of a volume that surrounds the tissue into which therapy signals are to be delivered. An outer surface of the cylindrical image the tissue can be displayed on a two-dimensional display as a strip 212 made by cutting the cylindrical image along a virtual line 214 and “unrolling” the perimeter of the cylindrical image for display on a two-dimensional screen. If no gas, bowel tissue, bone or other non-desired tissue is visible within the cylindrical image, then it is likely that no bowel or gas is in the path of the therapy beam. The illumination signals 200 used to produce the cylindrical image can be produced by the therapy transducer or by selected elements of an annular imaging array. As indicated above, higher power piston elements may be incorporated into the annular imaging array and use to increase the signal to noise ratio of the corresponding echo signals.

FIG. 3C shows an example of a conical image 220 that can be produced by orienting the receive elements of an annular imaging array such that they view the outside of the volume into which therapeutic signals are to be delivered. A conical image 220 is similar to a cylindrical image shown in FIG. 3B except that the proximal and distal diameters of the tissue included in the image are different. Therefore, as used herein, a conical image is considered to be a special type of cylindrical image. An outer surface of the conical image can be displayed by cutting the conical image along a virtual line 222 and “unrolling” the perimeter of the cone for display on a two-dimensional screen. In some embodiments, the conical image includes the outer boundaries where the therapy beam is expected to pass.

In another embodiment of the disclosed technology, the annular imaging arrays can be used to detect the elasticity or other mechanical characteristic of tissue. When used in this manner, an illumination pulse is delivered to the tissue by the therapy or imaging transducer and corresponding echo signals are detected. Next a higher power “push” pulse is delivered to the tissue by the therapy transducer or an annular imaging array. Following the delivery of the push pulse, another lower power illumination pulse is delivered by the therapy transducer or the annular imaging array and corresponding echo signals are detected. A comparison is then made to the echo signals detected before the push pulse. The difference in signals (typically measured as a phase shift) is therefore a measure of relative motion of any given point within the tissue volume as a result of the push pulse. This relative motion can be used to calculate relative or absolute values of mechanical properties such as tissue strain, elasticity or stiffness, compressibility or Poisson's ratio. These mechanical characteristics of the tissue in the target volume may be used to determine when the tissue has been sufficiently treated, to identify elasticity or stiffness differences between tissues in the illumination space, or to identify tissue types (e.g. fibroids) based on a comparison against measurements made from known tissue types. The mechanical characteristics can be color coded and displayed to provide an indication of the characteristic value at each location.

As used herein, an “image” of the tissue is therefore intended to include conventional images of tissue such as B-mode images where each point in the tissue is represented by its echo intensity or power. The term image also includes representations where each point in the image encodes or represents a mechanical characteristic. The images may or may not be human perceptible. For example, the image may represent an array of data stored in a memory that is used by the computer system to control treatment without display on a screen for the user. The illumination signal can therefore be used to create all these types of images. In addition illumination signals at high power from the therapy transducer can be used to treat the tissue.

FIG. 4A illustrates yet another embodiment of the disclosed technology to increase the received signal to noise ratio by improving the transmit sensitivity. In this embodiment, an imaging array includes a number of elements that are displaced a distance, d, from the skin surface. If an element of an annular imaging array is displaced away from the skin surface, then the power at which it is operated can be increased because the signal disperses or spreads out by the time it reaches the skin. However the area exposed to the illumination signal increases as well thereby delivering more illumination power into the viewing space. For example, if the maximum energy permitted at a skin surface is 500 milliwatts per square centimeter, the element may be operating at higher power, say 600 milliwatts. The power at the skin surface is still 500 milliwatts due to the dispersion but the area exposed is greater than if the element were directly against the skin.

FIG. 4B shows an alternative embodiment of an annular imaging array where the elements are positioned against the skin surface. In this case a group of elements 260 are operated to simultaneously transmit illumination signals. If several elements are used simultaneously, more energy is applied to the tissue in the viewing space. In order for the illumination signal to illuminate the tissue evenly as with a small, single element, the energy should appear as if it is coming from a single point source. Therefore the elements of the group 260 are mechanically (e.g. shaped or lensed) or electrically focused so that the signals appear to come from a single point source 262 that is behind the group or from a single point source 264 that is in front of the combined elements.

To produce a fully synthetic aperture image (e.g. transmit and receive), illumination signals are produced at each element of one of the annular imaging arrays (e.g. the outer annular imaging array) and echo signals are detected from each element of the other of the annular imaging arrays. The results are stored in a matrix or other suitable arrangement, and processed to produce the synthetic aperture image. As will be appreciated, in an alternative design the elements of the inner annular imaging array are focused or lensed to provide a virtual point source while the elements of the outer annular imaging array are used to receive the echo signals. Because the point sources of the illumination signals are virtual and not located against the skin, greater signal power can be applied

While illustrative embodiments have been illustrated and described, it will be appreciated that various changes can be made therein without departing from the spirit and scope of the invention. For example, although the annular imaging arrays are shown as being circular, it will be appreciated that such annular imaging arrays could be formed of strips of linear arrays to form an open or closed polygon around the therapy transducer. Similarly, although the disclosed embodiments of the applicator use one or two annular imaging arrays, additional annular imaging arrays could be included to aid in transmission or receipt of ultrasound signals. It is therefore intended that the scope of the invention be determined from the following claims and equivalents thereof.

The embodiments of the invention in which an exclusive property or privilege is claimed are defined as follows:

1. A system for treating and imaging tissue in a body, comprising:
a therapy transducer that is operable to selectively deliver therapeutic ultrasound signals to a target treatment volume in the body or to deliver illumination signals into a viewing space within the body;
a multi-element imaging array including a number of receive elements surrounding the therapy transducer;
a transmit controller configured to control the application of driving signals to the therapy transducer in order to produce an illumination signal for delivery into the viewing space;
a receive controller configured to receive signals from the elements of the multi-element imaging array that are created in response to tissue being exposed to the illumination signal produced by the therapy transducer; and
a processor configured to combine the received signals in order to produce an image of tissue in the viewing space.

2. The system of claim 1, wherein the receive elements of the multi-element imaging array are arranged in an annular array and wherein each receive element has at least one dimension that is less than the wavelength of the illumination signal.

3. The system of claim 1, wherein the transmit controller is configured to control the application of driving signals to the therapy transducer such that the illumination signal is transmitted at a therapeutic power level.

4. The system of claim 1, wherein the transmit controller is configured to control the application of driving signals to the therapy transducer such that the illumination signal is transmitted at a power level that is less than a therapeutic power level.

5. The system of claim 1, wherein the transmit controller is configured to control the application of driving signals to the therapy transducer such that therapeutic ultrasound signals and the illumination signals from the therapy transducer have different frequencies.

6. The system of claim 1, wherein the multi-element imaging transducer is an annular ring that surrounds the therapy transducer.

7. The system of claim 1, wherein the image represents the echo intensity of a number of locations within the area illuminated by the illumination signal.

8. The system of claim 1, wherein the image represents a mechanical characteristic of tissue at a number of locations within the area illuminated by the illumination signal.

9. The system of claim 1, wherein the therapy transducer is controllable to sequentially direct illumination signals throughout the viewing space.

10. The system of claim 1, wherein the therapy transducer is controllable to simultaneously direct illumination signals throughout the viewing space.

11. An applicator for treating and imaging tissue in a body, comprising:
a therapy transducer configured to deliver therapeutic ultrasound signals to a target treatment volume of tissue in the body;
an imaging array including one or more receive elements surrounding a perimeter of the therapy transducer; and
one or more driving elements positioned around the perimeter of the therapy transducer that are larger than the one or more receive elements and are configured to supply illumination signals of sufficient acoustic power into a viewing space such that the one or more receive elements in the annular imaging array can detect acoustic echo signals.

12. The applicator of claim 11, wherein the one or more driving elements and the one or more receive elements are in the same array.

13. The applicator of claim 11, wherein the one or more driving elements and the one or more receive elements are in separate arrays.

14. The applicator of claim 11, wherein the one or more driving elements are mechanically movable around the therapy transducer.

15. The applicator of claim 11, wherein the one or more receive elements are mechanically movable around the therapy transducer.

16. The applicator of claim 11, wherein applicator is connectable to a processor that is configured to produce an image of the tissue at one or more locations in the body, wherein the image represents the echo intensity of a number of locations within the area illuminated by the illumination signal.

17. The system of claim 11, wherein the applicator is connectable to a processor that is configured to produce an image of the tissue at one or more locations in the body, wherein the image represents a mechanical characteristic of tissue at a number of locations within the area illuminated by the illumination signal.

18. The system of claim 11, wherein driving signals applied to the one or more driving elements include spatial or temporal coding.

19. The system of claim 11, wherein the one or more driving elements are focused to produce illumination signals that appear to originate at a point source that is displaced from a front surface of the driving elements.

20. The system of claim 11, wherein the one or more driving elements are displaced from a tissue surface when the applicator is placed on a body.

21. A system for treating and imaging tissue in a body, comprising:
a therapy transducer that is configured to deliver therapeutic ultrasound signals to a target treatment volume of tissue in the body;
an illumination source that is configured to deliver illumination signals to a viewing space in the body;
an imaging array including one or more receive elements surrounding the therapy transducer, wherein the one or more receive elements are oriented to detect signals from a cylindrical volume within the viewing space;
a transmit controller configured to control the illumination source to deliver the illumination signals into the viewing space;
a receive controller configured to receive signals from the elements of the multi-element imaging array that are created in response to tissue being exposed to the illumination signals; and
a processor configured to combine the received signals in order to produce a cylindrical image of tissue in the viewing space.

22. The system of claim 21, further comprising a display, wherein the processor is configured to produce an image of an outer surface of the cylindrical image as a strip on the display.

23. The system of claim 21, wherein the cylindrical image is a conical image.

24. The system of claim 23, further comprising a display, wherein the processor is configured to produce an image of an outer surface of the conical image on the display.

25. The system of claim 21, wherein the illumination source is the therapy transducer.

26. The system of claim 21, wherein the imaging array includes one or more higher power driving elements that are used as the illumination source.
